(19) United States
(12) Patent Application Publication
(10) Pub. No.: US 2022/0395546 A1
(43) Pub. Date: Dec. 15, 2022

(54) METHODS AND COMPOSITIONS FOR THE EXTRACTION OF PHYTOCHEMICALS FROM PLANT MATERIAL

(71) Applicant: eHempHouse Corp., Binghamton, NY (US)

(72) Inventors: Andrew T. Neal, Pleasant Valley, NY (US); Adosh Mehta, New City, NY (US)

(21) Appl. No.: 17/345,923

(22) Filed: Jun. 11, 2021

(51) Int. Cl. A61K 36/185; A61K 31/05; A61K 31/352; A61K 47/44

(52) U.S. Cl. CPC A61K 36/185; A61K 31/05; A61K 31/352; A61K 47/44; A61K 2236/15; A61K 2236/37

(57) ABSTRACT

This disclosure describes extraction methods that allow for the release of large amounts of phytochemicals (e.g., cannabinoids) into a lipid phase, resulting in a high yield method, even in the absence of any organic solvents. As described herein, a naturally occurring lipid phase (e.g., from oilseeds) can be used.

### TECHNICAL FIELD

This disclosure generally relates to methods and compositions for the extraction of cannabinoids from Cannabis.

### BACKGROUND

There are over 100 cannabinoids in Cannabis, including cannabidiol (CBD) and tetrahydrocannabinol (THC). Cannabinoids have been shown to be structurally similar to chemicals that the human body makes that are involved in appetite, memory, movement, and pain. Limited research with cannabinoids suggests that cannabinoids may be effective in killing cancer cells and slowing tumor growth, controlling nausea and vomiting (e.g., caused by cancer chemotherapy), reducing anxiety, reducing inflammation and relieving pain, relaxing tight muscles (e.g., in people with MS), and stimulating appetite and improving weight gain (e.g., in people with cancer and AIDS). In addition, cannabinoids have been effective in reducing seizures, sometimes dramatically, in individuals, and the FDA recently approved EPIDIOLEX®, the active ingredient of which is cannabidiol (CBD), as a therapeutic for individuals with severe or hard-to-treat seizures.

### SUMMARY

The extraction methods described herein allow for the release of large amounts of phytochemicals (e.g., cannabinoids) into a lipid phase, resulting in a high yield method, even in the absence of any organic solvents. As described herein, a naturally occurring lipid phase (e.g., from oilseeds) can be used.

In one aspect, methods of obtaining cannabinoids from Cannabis biomass are provided. Such methods typically include combining seed from a plant (e.g., an oil seed plant) and Cannabis biomass, wherein the seed comprises at least 14% by weight oil (e.g., at least 16%, 17%, 18%, 19%, 20%, or 22% by weight), wherein the seed and the biomass are combined at a ratio of about 2:1 to about 8:1 (e.g., about 3:1 to about 6:1; about 4:1 to about 5:1); and compressing the seed and the biomass under conditions where the oil from the seed is released and the cannabinoids from the Cannabis biomass are released, thereby obtaining cannabinoids from the Cannabis biomass.

In some embodiments, the seed from a plant is seed from a Cannabis plant, a sunflower plant, or a canola plant. In some embodiments, the oil is hemp oil, sunflower oil, canola oil, coconut oil, or grapeseed oil.

In some embodiments, the compressing step comprises using a screw press or a hydraulic press. In some embodiments, the screw press is an expeller screw press. In some embodiments, the screw is a continuous screw.

In some embodiments, the compressing step is performed at a temperature of about 160° F. to about 200° F. (e.g., about 175° F. to about 190° F.; about 180° F. to about 185° F.).

In some embodiments, the method further includes reducing the size of the biomass prior to the combining step. In some embodiments, the biomass has an average particle size of about 2 mm to about 10 mm. In some embodiments, the biomass comprises predominantly flowers and leaves. In some embodiments, the biomass comprises little to no stem/stalk material. In some embodiments, the biomass comprises trichomes.

In some embodiments, the method further includes adding oil (before, during or after) the compressing step. In some embodiments, the oil is from the same type of plant. In some embodiments, the oil is from a different type of plant.

In some embodiments, organic solvents are not added.

In another aspect, cannabinoids produced by the method described herein are provided. In some embodiments, the cannabinoids are essentially free of organic solvents (e.g., hexane, butane, alcohols, etc.). In some embodiments, the cannabinoids are not significantly modified (e.g., non-decarboxylated). In some embodiments, the cannabinoids include full spectrum cannabinoids.

In still another aspect, cannabinoid compositions are provided that include essentially no (or trace amounts or less of) organic solvent; and full spectrum cannabinoids (e.g., little to no modified (e.g., decarboxylated) cannabinoids).

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION

The current disclosure describes methods of extracting plant-derived chemicals, also referred to as phytochemicals, using oil as the solvent and carrier. Most conventional extraction processes used to obtain phytochemicals use organic solvents such as hexane, butane, or isopropanol to chemically extract the desirable phytochemicals, followed by a concentration step (e.g., distillation) to remove the organic solvents. Organic solvents, however, can be toxic, and the steps required to remove them can be costly, wasteful, unfriendly to the environment, and often involve elevated temperatures, which can affect and alter the chemical structure of the desired phytochemicals.

In the extraction methods described herein, the oil serves multiple purposes. First, the oil acts as the solubilizing carrier medium in which the desirable phytochemicals, which typically have very limited solubility in polar (aqueous) solvents, are extracted and concentrated. Using oils eliminates the need to use, and then remove, organic solvents. In addition, the oil regulates the temperature during extraction by functioning as a heat transfer medium, dissipating the heat generated during the process and keeping the operating temperatures at a level that allows for the full spectrum of desired phytochemicals to be extracted without the damaging effects of heat.

A suitable oil for use in the extraction methods described herein can include, without limitation, soybean oil, olive oil, sunflower oil, canola oil, hemp oil, or grapeseed oil, coconut oil. As described herein, the oil used in the extraction methods described herein can be a naturally occurring lipid phase found, for example, in plants such as oilseed plants. Any type of seed that has a suitable oil content can be used, and representative plants that contain a suitable naturally occurring lipid phases, include, without limitation, sunflower, canola, hemp, and grape.

In an exemplary embodiment, the methods and compositions described herein are applied to the extraction of cannabinoids from Cannabis plants (e.g., Cannabis sativa). As used herein, cannabinoids refers to any of the more than 100 chemical compounds identified in Cannabis plants including the most common, cannabidiolic acid (CBDA) and tetrahydrocannabinolic acid (THCA), as well as the decarboxylated forms of each, cannabidiol (CBD) and tetrahydrocannabinol (THC), respectively, as well as, for example, cannabigerolic acid (CBGA) and cannabigerol (CBG). The methods and compositions described herein, however, can be applied to any number of phytochemicals from any number of plants.

As described herein, a suitable amount of a naturally occurring oil is combined with a suitable amount of plant biomass that contains the desired phytochemicals.

Although naturally occurring oil that previously was extracted from the seed of a plant can be used in the methods described herein, significantly, the extraction methods described herein have been designed to accommodate plant seed as the source of the naturally occurring oil. Therefore, a suitable amount of plant seed (i.e., an amount of plant seed containing a suitable amount of oil) can be used in the methods described herein. Plant seed suitable for use in the methods described herein typically have an oil content of at least 14% (by weight) (e.g., an oil content of at least 15%, 16%, 17%, 18%, 19%, 20%, 21% or 22% by weight) but plant seed having a higher oil content (e.g., hemp seed can be as high as 35% oil content; sunflower seed can be as high as 60% oil content) can be used in the methods described herein. It would be appreciated that the ratio of seed to biomass can be adjusted when using seeds having a very high oil content. If, for example, there is insufficient seed or the seed contains an insufficient amount of oil, it would be appreciated that other forms of naturally occurring oil can be used in the extraction methods described herein to supplement the oil originating from the plant seed. Oil can be added to the combination of plant seeds and plant biomass before, during and/or after the material is compressed, and the oil can be from the same type of plant or a different type of plant as the seed.

The particular plant biomass that is used in the extraction methods described herein will largely depend upon where the desired phytochemicals are localized in the plant. In Cannabis, the cannabinoids are contained in, and secreted from, trichomes, which are located predominantly in the flower buds. Therefore, it would be desirable for the Cannabis biomass to be predominantly flowers (e.g., to maximize the presence of high-yield plant material, e.g., trichomes), and it also would be desirable for the Cannabis biomass to contain little to no stem and/or stalk material (e.g., to minimize the presence of low-yield plant material).

While intact flower buds can be used as the plant biomass in the methods described herein, it may be beneficial (e.g., to increase yield) to manually or mechanically reduce the size of the flower buds into smaller biomass material. There are many different methods available for reducing the size of plant material, and the method by which the plant material is reduced is not as important as the size of the resulting plant material. For example, the size of Cannabis biomass can be reduced by chopping or grinding, and Cannabis biomass having an average particle size that ranges from about 2 mm to about 10 mm (e.g., about 3 mm, about 4 mm, about 5 mm, about 6 mm, about 7 mm, about 8 mm, or about 9 mm) can be used in the methods described herein. It would be understood that the size of the plant biomass can be reduced prior to being combined with the plant seed, after being combined with the plant seed, or both.

The ratio of plant seed to plant biomass can be modified to control the efficiency and resulting yield of the phytochemicals. For example, a seed to biomass ratio of from about 2:1 up to about 8:1 (e.g., from about 2:1 up to about 6:1, or from about 2:1 up to about 4:1) results in effective extraction of phytochemicals from plant biomass material. In some embodiments, the ratio of seed to biomass used is about 3:1. The particular ratio of seed to biomass used in the methods described herein can depend upon, for example, the amount of naturally occurring oil that is present in the seed, the particle size of the biomass the chemical make-up of the phytochemicals, and the moisture content of the seeds and/or the biomass (a moisture content of about 5% to about 10% is desired).

Once the naturally occurring oil (e.g., in the form of plant seeds) and the plant biomass have been combined at a suitable ratio, the material is compressed so as to release the oil contained within the seed. Devices for applying compression to plant material typically use a shear force (e.g., generated by a screw press or a hydraulic press having, e.g., an expeller design) to disrupt the hard coat of the seed and the cell walls of the tissue within. Compression of the seed allows for the oils inside to be liberated. In addition, the shear force generated by compression of the plant material also causes separation within the biomass material (in Cannabis, e.g., the trichomes are released). Further, the frictional heat generated during the compression process (e.g., due to the shearing action) also helps release phytochemicals (e.g., in Cannabis, e.g., from the resins in the trichomes) and they become solubilized in the oil that is released from the accompanying seeds in the mixture.

The temperature at which the compression and/or shearing is performed can have a significant effect on the yield and purity of the resulting phytochemical as well as the extent to which the phytochemicals are modified. The temperature at which the extraction methods described herein is carried out can be carefully controlled and can be kept low enough to prevent any chemical transformation (e.g., decarboxylation) and/or degradation of the phytochemicals. In the exemplary embodiment for obtaining cannabinoids, representative temperatures at which the extraction can take place include, without limitation, a temperature of about 160° F. to about 200° F. (e.g., about 175° F. to about 190° F.; about 180° F. to about 185° F.). It would be appreciated that, in certain preparations of cannabanoids, it is desirable to have little to no modification (e.g., decarboxylation) of the cannabinoids; in other preparations, the extent of modification may not be important or may be desirable.

The methods described herein can be scaled up for commercial scale production of one or more cannabinoids (e.g., CBD). On a commercial scale, an exemplary system can include, without limitation, the following components.
Hammer Mill: a device used to reduce the size of the Cannabis plant biomass prior to entering the press.
Feed Hopper: a device used to hold the feed. As described herein, the feed includes a mixture of seed and Cannabis plant biomass in a particular ratio. The ratio of seed to biomass is one of the parameters that can be altered as described herein.
Screw Press: a screw (e.g., a continuous screw made of hardened and polished stainless steel) powered by a motor coupled to a reducer gear assembly controlled by a variable frequency drive. The speed of the motor during crushing is one of the parameters that can be altered as described herein.
Heating Collar: a device used to maintain a constant preset temperature in the crush barrel of the screw press. The heating collar typically includes a temperature controller and a thermocouple.
A Collection Device: a container for collecting the oil from the crushed seed.
A Settling Tank: a container, usually connected to a filtration device, used to remove particulate matter (e.g., plant material) or other unwanted materials from the oil.
A Collection Bin: a container for collecting the pressed seed meal.

It would be appreciated by a skilled artisan that a number of the components described above have equivalents that are capable of performing the same or similar function, sometimes referred to as “functional equivalents.”

The methods described herein result in the production of a phytochemical-enriched (e.g., cannabinoid-enriched) oil. Since the methods described herein are performed at a low temperature, the phytochemicals can be preserved without altering their chemical structure. Accordingly, the methods described herein is able to obtain full spectrum cannabinoids. As used herein, full spectrum refers to cannabinoids that are predominantly in the acidic form (e.g., cannabidiolic acid (CBDA) and tetrahydrocannabinolic acid (THCA)) with little to no decarboxylated forms (e.g., cannabidiol (CBD) or tetrahydrocannabinol (THC)). The methods described herein can use any type of plant (e.g., for Cannabis, hemp plants as well as marijuana plants) as the starting biomass. In addition, the extracting medium can be varied, simply by selecting different oil rich seeds.

The methods described herein result in the production of a phytochemical composition that contains essentially no organic solvent (e.g., hexane, butane, alcohols, etc.). As used herein, essentially no organic solvent refers to the absence of any organic solvent or the presence of only trace amounts in the final phytochemical product. As used herein, trace amounts typically refers to less than 2% (by weight) (e.g., less than 1.5%, 1.0%, 0.5%, 0.2%, 0.1%, or 0.01% (by weight)) as determined, for example, using a suitable GC/MS analysis. The methods described herein also produce a cannabinoid composition that contains full spectrum cannabinoids (e.g., cannabinoids with little to no modifications (e.g., decarboxylation)). For example, cannabinoids produced by the methods described herein can contain significant amounts of the acid forms of such compounds (e.g., CBDA, THCA, CBGA) and little to no amounts of the decarboxylated forms of such compounds (e.g., CBD, THC, CBG, respectively). As used herein, “little to no” refers to less than 2% (by weight) (e.g., less than less than 1.5%, 1.0%, 0.5%, 0.2%, 0.1%, 0.01%, or 0.001% (by weight)) as determined, for example, using a suitable chromatographic assay such as, without limitation, Thin Layer Chromatography (TLC), High Performance Thin Layer Chromatography (HP-TLC), Gas Chromatography-Mass Spectrometry (GC-MS), or High Performance Liquid Chromatography-Mass Spectrometry (HPLC-MS).

Significantly, the primary byproduct resulting from the methods described herein is a high-protein seed meal cake that can be used as a food product (e.g., for humans) or an animal feed product (e.g., for companion animals, exotic/zoo animals, and farm animals). On the other hand, the seed meal resulting from current methods is unusable waste matter since it is infiltrated with organic solvents.

As discussed above, the extraction methods exemplified herein are not limited to only obtaining cannabinoids from Cannabis biomass. Any number of other phytochemicals can be obtained from all types of plant material using this method simply by optimizing the process parameters described above.

In addition to obtaining highly purified, full spectrum cannabinoids, the extraction methods described herein can be used to infuse an oil (e.g., a naturally occurring oil) with cannabinoids or other phytochemicals such as, without limitation, anti-oxidants, flavonoids, phenolic compounds, or combinations thereof.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Representative Extraction Using Seed

Biomass was provided by Danny Shelton (Knoxville, Tenn.) or Hempire State Growers (Milton, N.Y.). To produce the biomass that was used in the following experiments, stalks were removed from dried hemp, and the flower and leaf biomass was collected and coarsely chopped in a food processor to roughly twice the size of the hemp seed being used.

6 lbs of the coarsely chopped biomass (from variety ‘Merlot’), which has a CBD content of about 11% was used, along with 18 lbs of hemp seed (from variety ‘Legacy’), which has an oil content of about 20% (Legacy Hemp, S.D.).

The seed material was introduced into a M70 screw press (Oil Press Co., Mondovi, Wis.) in the absence of a die. Once packed, the press was activated at 50 Hz and dies were added (0.5 each) until the heat reached about 165 F. The biomass was added at this point, and the die size was decreased as much as possible (until the spent press cake is as dry as possible and the screw press is still operating). The combined material was compressed under the following conditions:
Temperature: varied between 175° F. and 195° F. in different experiments to examine effects on yield;
Start Temp: 135° F.
Stop Temp: 135° F.
Size (diameter)/type of screw: 75 mm diameter, 200 mm length
Die: 0.469″ orifice
Rate/Speed: about 30-35 hz

The combined material was pressed for at least one hour, and about 1 L of the oil, containing the cannabinoids, was produced. Roughly about 20-22 lbs of spent press cake was produced from the above-indicated biomass.

If desired, the spent press cake can be re-pressed by combining with the appropriate amount of seeds and repeating the procedure described above.

Following extraction, the recovered oil containing the cannabinoids is left in the container for about 4-6 hours to let any particulate matter settle, and then the oil was filtered through a M504 filter or a M504 filter followed by a MC55C (or MC55) filter to remove any waxy residue and any small particulate matter. The MC55C filters also contain an activated carbon layer that removed the chlorophyll and decolorized the oil.

This procedure has been used to produce about 300 gallons of oil to date, containing highly pure quantities of CBD and other cannabinoids.

### Example 2

### Exemplary Extraction Using Oil

Hemp oil was obtained from 50 lbs of seed using a M70 screw press.

Dry Cannabis biomass was coarsely chopped as described above, and then mixed with the hemp oil at a ratio of 1:3.4 (by weight).

The combined material was introduced into the screw press in the absence of a die until the desired temperature was reached, at which time the dies were added.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of obtaining cannabinoids from Cannabis biomass, comprising:
combining seed from a plant (e.g., an oil seed plant) and Cannabis biomass, wherein the seed comprises at least 14% by weight oil (e.g., at least 16%, 17%, 18%, 19%, 20%, or 22% by weight), wherein the seed and the biomass are combined at a ratio of about 2:1 to about 8:1 (e.g., about 3:1 to about 6:1; about 4:1 to about 5:1); and
compressing the seed and the biomass under conditions where the oil from the seed is released and the cannabinoids from the Cannabis biomass are released,
thereby obtaining cannabinoids from the Cannabis biomass.

2. The method of claim 1, wherein the seed from a plant is seed from a Cannabis plant, a sunflower plant, or a canola plant.

3. The method of claim 1, wherein the oil is hemp oil, sunflower oil, canola oil, coconut oil, or grapeseed oil.

4. The method of claim 1, wherein the compressing step comprises using a screw press or a hydraulic press.

5. The method of claim 4, wherein the screw press is an expeller screw press.

6. The method of claim 4, wherein the screw press uses a continuous screw.

7. The method of claim 1, wherein the compressing step is performed at a temperature of about 160° F. to about 200° F.

8. The method of claim 1, further comprising reducing the size of the biomass prior to the combining step.

9. The method of claim 8, wherein the biomass has an average particle size of about 2 mm to about 10 mm.

10. The method of claim 1, wherein organic solvents are not added.

11. The method of claim 1, wherein the biomass comprises predominantly flowers and leaves.

12. The method of claim 1, wherein the biomass comprises little to no stem/stalk material.

13. The method of claim 1, wherein the biomass comprises trichomes.

14. The method of claim 1, further comprising adding oil during the compressing step.

15. The method of claim 14, wherein the added oil is from the same type of plant as the seeds.

16. Cannabinoids produced by the method of claim 1.

17. The cannabinoids of claim 16, wherein the cannabinoids are essentially free of organic solvents.

18. The cannabinoids of claim 16, wherein the cannabinoids are not significantly decarboxylated.

19. The cannabinoids of claim 16, wherein the cannabinoids comprises full spectrum cannabinoids

20. The cannabinoids of claim 16, wherein the cannabinoids comprises cannabinol and tetrahydrocannabinol (THC).

21. A cannabinoid composition, wherein the composition comprises:
essentially no organic solvent; and
full spectrum cannabinoids.
